(19) United States
(12) Patent Application Publication
(10) Pub. No.: US 2026/0151093 A1
(43) Pub. Date: Jun. 4, 2026

(54) DETERMINING A RISK FOR A MACE EVENT

(71) Applicant: Beckman Coulter, Inc., Brea, CA (US)

(72) Inventors: Sean M. Roberts, Brea, CA (US); Girish Simon, Brea, CA (US); Maxim Shatsky, Brea, CA (US); Rui Hu, Brea, CA (US)

(21) Appl. No.: 19/138,045

(22) PCT Filed: Dec. 7, 2023
(86) PCT No.: PCT/US2023/082857

§ 371 Date: Jun. 11, 2025

Related U.S. Application Data
(60) Provisional application No. 63/386,949, filed on Dec. 12, 2022.

(51) Int. Cl. A61B 5/00; A61B 5/0205; G06N 20/00; G16H 10/60; G16H 40/63

(52) U.S. Cl. CPC A61B 5/7275; A61B 5/0205; A61B 5/7267

(57) ABSTRACT

A computer-implemented method of determining, for a patient, a risk of developing a major adverse cardiovascular event. MACE, is described. The method comprises obtaining, at a computing device, a set of patient data indicative of a) at least one troponin value of the patient, b) an age of the patient, and c) at least one of a respiratory rate value of the patient and an anion gap value of the patient. The method further comprises determining, based on processing the obtained set of patient data with the computing device, at least one MACE score indicative of the risk for a MACE event occurring at the patient.

### CLAIM OF PRIORITY

This application claims the benefit of priority of U.S. Provisional Application Ser. No. 63/386,949, filed Dec. 12, 2022, which is hereby incorporated by reference in its entirety.

### TECHNICAL FIELD

The present disclosure generally relates to the field of clinical decision support. In particular, the present disclosure relates to a computer-implemented method of determining a patient-specific risk of developing a major adverse cardiovascular event (referred to herein as “MACE”). Further, the present disclosure relates to a computer program for carrying out steps of said method, to a computer-readable medium storing such computer program, and to a computing device or system configured to perform steps of said method.

### BACKGROUND

An increasing number of patients or individuals is assessed in hospitals for health-relevant cardiac events, which typically includes an assessment for a likelihood of a potential or future major adverse cardiovascular event (MACE) occurring at a patient, for example at or within a certain period of time after the assessment. Typically, patients diagnosed or assessed at a hospital for an emergent health-relevant cardiac event or MACE, for example, can be suspected of having an acute coronary syndrome (also referred to herein as “ACS”), a range of serious conditions involving the heart, for example, myocardial injury, ischemic cardiovascular events, heart failure, decompensated heart failure, myocardial infarction, myocardial or pericardial infection. Therein, the risk of MACE to occur in or at a patient typically refers toa probability or likelihood for MACE, i.e. for a potential adverse development or future outcome. Thus, the risk or likelihood for MACE, for example, can be a measure of a safety profile for discharging a patient from a hospital or emergency department. In particular, the likelihood, probability or risk of a potential negative or health-relevant event (or MACE) can be used at a hospital to decide to discharge a patient or to admit them for further examination, for example into an ACS pathway for further diagnostic and/or therapeutic treatment.

In some instances, the risk of MACE can also be evaluated after an assessment for, or diagnosis of, a health-relevant cardiac event. Accordingly, a patient may be evaluated for a health-relevant cardiac event at a hospital or emergency department, and the risk or likelihood of MACE for the patient can additionally be determined, evaluated and/or prognosed. In this context, the risk or likelihood for MACE can be considered as a prognostic risk or likelihood of an adverse or negative outcome of a health-relevant cardiac event potentially occurring or developing in a patient. Optionally, the risk of MACE can be determined for a certain time period. For example, the risk of MACE may be determined for a certain period of time after the assessment of the cardiac event at the hospital, such as within 7 days of the assessment of the cardiac event, within 30 days of the assessment of the cardiac event at the hospital, within 60 days of the assessment of the cardiac event at the hospital, or within 90 days of the assessment of the cardiac event at the hospital.

Generally, the term MACE can be used to describe or comprises a variety of different types of adverse health events of cardiac origin, such as one or more of acute coronary syndrome conditions or related negative health events such as, myocardial injury, cardiovascular death, ischemic cardiovascular events, heart failure, myocardial infarction, and/or need for urgent revascularization. In particular, all types of acute myocardial infarction (AMI) including ST-elevation myocardial infarction (STEMI) and Non-ST-elevation myocardial infarction (NSTEMI), as well as heart-related death can be referred to as MACE event. Further, Coronary Artery Bypass Graft (CABG) surgery, cerebral infarction, Percutaneous Coronary Intervention (PCI), nontraumatic hemorrhage, death, stent procedure, and re-stent procedure can also be referred to as MACE events. Moreover, a new onset of heart failure in addition to one or more other MACE events events can be referred to as further MACE event.

Patients assessed for a health-relevant cardiac event, MACE and/or ACS at a hospital or emergency department typically report chest pain, shortness of breath, and often additional symptoms suggestive of cardiac involvement. The standard test for diagnosing a health-relevant cardiac event among suspect ACS patients at hospitals includes a troponin (Tn) assay and electrocardiogram data, for example used in conjunction with current and/or past clinical data, to determine if a patient is suffering cardiac injury. High-sensitivity troponin testing may aid with the risk stratification of patients assessed for ACS, including the risk for MACE. These high-sensitivity troponin assays can detect lower levels of troponin in the blood with analytical sensitivities up to 100 times greater than conventional troponin assays. Use of high-sensitivity troponin assays can therefore enable the detection of small changes in troponin levels or values and can help identify patients exhibiting cardiac injury or high risk for MACE (also referred to as “cardiac patients”) and patients unlikely to be exhibiting cardiac injury or developing MACE (also referred to as “non-cardiac patients”), for example to help triage patients more accurately and rapidly. Although the troponin level may aid in the diagnosis of acute myocardial infarction in some patients, it may be challenging to accurately risk stratify a patient, for example rule in or rule out a patient as having a high risk for MACE or as being at risk for MACE based on the troponin level or value alone.

### SUMMARY

It may therefore be desirable to provide for an improved method and device for reliably and accurately determining, for a patient, a risk, probability, and/or likelihood of developing MACE.

This is achieved by the subject matter of the independent claims. Exemplary embodiments are incorporated in the dependent claims and the following description.

Aspects of the present disclosure relate to a computer-implemented method of determining and/or assessing, for a patient, subject or individual, a risk of developing MACE, to a computer program for carrying out steps of said method, to a computer-readable medium, for example a non-transitory computer-readable medium, storing such computer program, and to a computing device or system configured to perform steps of said method. Any disclosure presented herein with reference to one aspect of the present disclosure equally applies to any other aspect of the present disclosure.

According to an aspect of the present disclosure, there is provided a computer-implemented method of determining for a patient, a risk of developing a major adverse cardiovascular event, MACE. Alternatively or additionally, the method may relate to a computer-implemented method of determining a patient-specific risk of suffering from MACE, of developing MACE and/or of experiencing MACE. The method comprises obtaining, at a computing device or system, a set of patient data indicative of and/or including a) at least one troponin value of the patient, b) an age of the patient, and c) at least one of a respiratory rate value of the patient and an anion gap value of the patient. The method further comprises determining, calculating and/or computing, based on processing the obtained set of patient data with the computing device, at least one MACE score indicative of the risk for a MACE event occurring in that or in said patient.

The inventors surprisingly found that taking at least one troponin value, the age, and at least one of the respiratory rate value of the patient and the anion gap value of the patient into consideration, the risk for the patient suffering or developing MACE can be more accurately and reliably determined, for example when compared to determining the risk for MACE based on the troponin value of the patient only. Accordingly, by means of the computer-implemented method described herein, risk stratification, for example at hospitals or emergency departments, can be significantly improved, as the method and device described herein may allow to efficiently, reliably, and accurately risk stratify a patient, for example rule in or rule out a patient as a cardiac patient, as being at risk for MACE and/or as having a high risk of developing MACE.

Further, determining the at least one MACE risk score can advantageously allow for a quantitative assignment of a health risk or risk status to the patient, such as for example a low or high risk for MACE. In particular, a number of false positives and/or a number of false negatives in determining or assessing whether a patient has a low or high risk for MACE, such as a risk below or above a threshold risk, can be advantageously reduced by means of the computer-implemented method described herein.

Moreover, by determining the risk for MACE and/or the MACE score (also referred to herein as MACE risk score) based on the at least one troponin value, the age, and at least one of the respiratory rate value of the patient and the anion gap value, a health state of the patient can be comprehensively examined or evaluated. As a consequence, the determination of the risk for MACE can be individualized to individual patients or patient sub-groups, which can further reduce the number of false positives and/or false negatives. Also, the time required for determining the risk of MACE can be significantly reduced. Accordingly, the present disclosure may significantly improve differentiation between patients having a high likelihood of experiencing a MACE event and those who are unlikely to experience a MACE event.

The present disclosure, therefore, can provide for an improved clinical decision support, for example allowing to efficiently, reliably and accurately risk stratify a patient as having a high or low risk for MACE, for example a risk above or below a threshold risk. For instance, aspects of the present disclosure may facilitate earlier discharge of patients from hospitals for patients having a low risk of MACE or being not at risk for MACE, and earlier intervention for those patients who are more likely to experience a MACE event or are at risk for MACE. In particular, unnecessary hospitalization of patients with non-critical disorders and anxiety among patients may be efficiently avoided or reduced. Also, certain procedures of cardiac workflows and protocols can be avoided at hospitals, which can result in a better patient experience, improved utilization of healthcare resources and cost savings, all while maintaining a high safety profile.

For instance, the method and device described herein can allow to reduce or avoid overcrowding at emergency departments. Also, the use of scarce healthcare resources can be optimized, such as observation beds, and diagnostic modalities, such as cardiac stress tests, angiograms and imaging studies. Further, the patient experience can be improved, for example by reducing the length of an emergency department stay. Alternatively or additionally, healthcare institution-wide quality metrics relating to the management of ACS or MACE patients can be improved, disruption to emergency department workflows can be reduced by minimizing user-provided inputs to compute the MACE risk score, without adding unnecessary alerts and alarms. Moreover, the documentation of risk assessment can be automated and the ability for shared decision-making with patients about diagnostic and treatment choices can be significantly improved.

Although not limited thereto, the present disclosure may be of particular advantage for determining and/or assessing the risk of MACE in patients, where the troponin level or value alone may not suffice to immediately and/or definitely risk stratify a patient as having a cardiac event or developing MACE, for example where the troponin level of the patient may fall into a so-called indeterminate range or zone of troponin values. Therein, the indeterminate range or zone of troponin values may refer to or denote a range of troponin values, based on which the patient cannot, or at least not with sufficient certainty or sufficiently high probability, be ruled in or out as being a cardiac patient or non-cardiac patient, this is as having a high risk or low risk for MACE. Patients having troponin values in an indeterminate range of troponin values may also be referred to herein as indeterminate patients or as patients belonging to the group of indeterminate patients. Although not limited thereto, aspects of the present disclosure may allow to improve or maximize the risk stratification capabilities in the indeterminate group of patients.

Determining the risk, for the patient, of developing MACE may refer to, include and/or be interchangeably used herein with prognosing MACE for the patient, determining a patient-specific risk for MACE, and/or determining the likelihood or probability for the patient to develop, experience or suffer from MACE or a MACE event, for example at the time of assessment or within a certain period of time after the assessment or determination of the risk. Accordingly, the risk of developing MACE, as used herein, may generally refer to or be indicative of a patient-specific likelihood or probability for MACE to occur or develop at a patient.

As used herein, determining the risk for MACE may include computing and/or assessing the risk for MACE. For instance, determining may generally relate to or include finding out or coming to a decision about by the riks for MACE, which may optionally include a corresponding reasoning or assessment of the risk, a calculation and/or a computation of the risk or MACE score. Alternatively or additionally, computing the risk for MACE may include determining based on mathematical means and/or calculation, which may be computer-aided, computer-assisted and/or computer-implemented. Alternatively or additionally, assessing the risk for MACE may include determining an importance, a significance, and/or value for the risk for MACE and/or the MACE score.

As used herein, obtaining the set of patient data may include receiving the set of patient data at the computing device. Alternatively or additionally, obtaining the set of patient data may include downloading, retrieving, and/or accessing the set of patient data by the computing device.

Further, the MACE score may be indicative, descriptive or reflective of the likelihood or probability for MACE to occur in the patient. In particular, the MACE score may refer to a numerical measure indicative of the determined risk, likelihood or probability for MACE to occur in the patient.

In a non-limiting example, the MACE score may be provided on an arbitrary scale ranging from a minimum value, for example zero or 0, to a maximum value, for example one or 100. Any other scale, including relative and absolute scales, can be used to represent the MACE score.

Optionally, the MACE score may be compared by the computing device to one or more predetermined threshold values for the risk for MACE. For example upon determining that the determined risk for MACE for the patient equals or exceeds the at least one threshold value, the patient may be classified as patient having a high risk for MACE or as being at risk for MACE. Alternatively or additionally, upon determining that the determined risk for MACE for the patient is below the at least one threshold value, the patient may be classified or considered as having a low risk for MACE or as being not at risk for MACE. Accordingly, the patient may be classified or assigned to at least one group or sub-group of a plurality of sub-groups or groups of patients based on comparing the at least one MACE score to at least one threshold value for the risk for MACE. Therein, classification using more than two patient groups, for example including indeterminate patients or other patient (sub-) groups, may also be used.

In an example, determining the MACE score may include generating information indicative of the determined risk of MACE for the patient. Alternatively or additionally, the generated information may be indicative of the likelihood or probability for the patient having a health-relevant cardiac event, developing MACE and/or having MACE. In an example, the generated information may include an estimate of whether the patient suffers from MACE or will suffer from MACE. Optionally, the computing device may determine or generate an output based on or including the MACE score and/or the generated information, which may optionally be displayed at a user interface of the computing device and/or stored at the computing device, for example in an electronic health record associated with the patient. Further optionally, the generated information may include a classification result, for example classifying the patient as having high or low risk for MACE based on the determined MACE score.

The set of patient data, also referred herein as patient data, may refer to electronically stored or storable data comprising one or more data elements indicative of one or more parameter values or subject values, which can include the at least one troponin value, the age, and at least one of the respiratory rate value and the anion gap value of the patient. Therein, the set of patient data may be stored or storable in any suitable data format and structure. For instance, the set of patient data may refer to or be indicative of at least a part of an electronic health record of or associated with the patient. Optionally, the set of patient data may include other data, such as for example one or more of personal data, demographic data, medical data, metabolic data, vital data, clinical data, and laboratory data associated with the patient. Any one or more of the aforementioned data can include one or both current data and historic data indicative of a prior medical history of the patient.

In an example, the set of patient data may be stored on a data storage of the computing device and/or on at least one external data source, for example an external data source communicatively coupled with the computing device via a communication interface or circuitry of the computing device. Alternatively or additionally, obtaining the set of patient data at the computing device may include receiving, retrieving and/or accessing, with the computing device, the set of patient data on the data storage and/or the external data source.

Generally, the at least one troponin value of the patient may be based on any type of one or more troponin tests or measurements performed on the patient. For example, at least one troponin value of the patient can relate to, be based on or be the result of any one or more of a troponin I test (TnI), a high sensitivity troponin I test (hs-TnI), a troponin T test (TnT), and a high sensitivity troponin T test (hs-TnT). The corresponding troponin value may define a level, concentration or range of troponin. For example, the troponin value may be given as concentration of troponin per volume blood, such as for instance in nanograms per milliliter or equivalent.

For example, the at least one troponin value of the patient can relate to or be based on a measurement of troponin performed on the patient, for example based on laboratory testing of sample material or a blood sample of the patient. For instance, the at least one troponin value can refer to a level or concentration of troponin in the patient's blood at the time of testing and/or at the time of determining or assessing the risk of MACE for the patient. Alternatively or additionally, the age of the patient, the respiratory rate value and/or the anion gap value may refer to parameter values at the time of testing of troponin and/or at the time of determining or assessing the risk of MACE for the patient. The same applies to any other parameter value described herein as being potentially considered for the determination of the patient-specific risk for MACE.

In an exemplary implementation, determining the at least one MACE score includes computing the risk for the MACE event occurring at the patient based on or using at least one machine learning model or algorithm of the computing device configured to receive and process at least a subset of the patient data as input data. For instance, the at least one machine learning model may be configured, for example trained, to receive, simultaneously or sequentially, the at least one troponin value, the age, and at least one of the respiratory rate value and the anion gap value of the patient, and to provide the MACE score or information indicative thereof as output. Optionally, the MACE score or information indicative thereof may be provided by the computing device along with other information, such as an uncertainty for the MACE score and/or contextual information for the MACE score or its determination.

For instance, the computing device may be configured to invoke the at least one machine learning model upon receipt of at least a subset or the entire set of patient data. Therein, the at least one machine learning model may refer to a trained machine learning model or algorithm, for example trained with a set of training patient data, which may include a troponin value, an age, and at least one of a respiratory rate value and an anion gap value of one or more reference patients. Accordingly, the training patient data may include the parameters the computing device or machine learning model is configured to process as inputs to compute the MACE score, and an expected or desired output of the computing device or machine learning algorithm for one or more of the reference patients, such as for example an expected or desired MACE score for one or more reference patients. The trained machine learning algorithm or model may, for example, be a trained gradient boosting algorithm, or any other artificial intelligence-based (AI-based) algorithm, such as an artificial neural network, a feed forward neural network and/or a deep neural network. Accordingly, the computer-implemented method described herein may at least partly be implemented as trained machine learning algorithm or artificial intelligence-based algorithm. Alternative implementations of the method, for example based on a decision tree, however, are possible and also encompassed by the present disclosure.

According to an embodiment, the computing device comprises a classifier or classifier circuitry, which may include or be a trained machine learning algorithm or model, a trained gradient boosting algorithm, or any other AI-based algorithm, model implemented in the computing device in one or both hard- and software. The classifier may be part of a control circuitry of the computing device, which may include one or more processors for data processing or may be implemented as separate classifier circuitry in the computing device, for example in the form of an integrated circuit and/or application specific integrated circuit.

As used herein, the anion gap value may be indicative of a difference between one or more positively charged ion species or electrolytes in the patient's blood and one or more negatively charged ion species or electrolytes in the patient's blood. The anion gap value may be indicative of an acidity or ion balance of the patient's blood. Hence, taking the anion gap value into consideration for the determination of the risk for MACE may allow to comprehensively evaluate the patient's health state, thereby allowing for a more accurate and reliable determination of the risk for MACE, and consequentially allow for an improved risk stratification.

For example, the anion gap value may be indicative of a difference between Sodium ion (Na⁺) concentration and the sum of Chloride ion concentration and Bicarbonate ion (Cl⁻+HCO₃⁻) concentration in the patient's blood. Accordingly, the anion gap value may be given as AG=Na⁺−(Cl⁻+HCO₃⁻). Alternatively, the anion gap value (AG) may be indicative of a difference between the sum of Sodium and Potassium ion concentrations (Na⁺+K⁺) and the sum of Chloride ion concentration and Bicarbonate ion (Cl⁻+HCO₃⁻) concentration in the patient's blood. Accordingly, the anion gap value (AG) may be given as AG=(K⁺+Na⁺)−(Cl⁻+HCO₃).

One or more of the Potassium ion concentration, the Sodium ion concentration, the Chloride ion concentration and the Bicarbonate ion concentration may be measured, for example using sample material obtained from the patient at or around the time of assessment or determination of the risk of MACE, and corresponding values may be processed by the computing device to compute the MACE score and/or determine the risk for MACE.

In an exemplary embodiment, the method comprises obtaining and/or receiving, at the computing device, a Sodium ion concentration value and at least one of a Chloride ion concentration value and a Bicarbonate ion concentration value of the patient, and computing, with the computing device, the anion gap value based on the received Sodium ion concentration value and at least one of the Chloride ion concentration value and the Bicarbonate ion concentration value of the patient. For instance, the computing device may be configured to compute the anion gap value based on subtracting the sum of the Chloride ion concentration value and the Bicarbonate concentration value from the Sodium ion concentration value.

According to an embodiment, the obtained set of patient data includes a gender value indicative of a gender or sex of the patient, and the at least one MACE score may be computed based on the gender value and/or taking the sex or gender of the patient into account. This may further improve accuracy of the predicted or determined risk for MACE.

According to an embodiment, the obtained set of patient data is indicative of both the respiratory rate value and the anion gap value of the patient. Accordingly, the at least one MACE score may be computed based on or using the at least one troponin value, the respiratory rate value and the anion gap value. This may further improve accuracy of the predicted or determined risk for MACE.

In an example, the obtained set of patient data may be indicative and/or include the at least one Troponin value, the age, the respiratory rate value, the anion gap value and the gender value, and the at least one MACE score may be computed based thereon.

Optionally, one or more of the following may be included in the set of patient data and considered for computing the at least one MACE score: an indicator for a history of Acute Myocardial Infarction (AMI) of the patient, a race or demographic value indicative of the race of the patient, a body temperature value, a white blood cell concentration value, a Calcium concentration value, a blood urea nitrogen (BUN) testing value, an erythrocyte mean corpuscular hemoglobin concentration value (MCHC), a Sodium ion concentration value, a Potassium ion concentration value, a Hematocrit value, a Creatine value, a red blood cell concentration value, a red cell distribution width value, a Hemoglobin value, a mean corpuscular volume value (MCV).

In an exemplary embodiment, the respiratory rate value, also referred to as breathing rate value, is indicative of a mean respiratory rate of the patient during a period of time around or about a time of determining the at least one troponin value of the patient, which may correspond to or differ from the time of assessment of the risk for MACE. For instance, the respiratory value may be determined within a predefined period of time around or about the time of determining the at least one troponin value. Therein, the predefined period of time can have a start time before, substantially equal to, or after the time of determining the at least one troponin value. Alternatively or additionally, the predefined period of time may have an end time before, substantially equal to or after the time of determining the at least one troponin value. The phrase “about or around the time of determining the at least Tn value”, may for example refer to a predefined time period of several minutes to several days, for example several hours, such as 1 to 10 hours, preceding, following and/or including the time or time instant of determining the Tn value.

Optionally, a plurality of respiratory rate values may be determined around or about the time of determining at least one troponin value, and/or may be received at the computing device, wherein the computing device may be configured to determine a mean respiratory rate value based on at least a subset of the plurality of received respiratory rate values. Therein, different respiratory rate values may refer to values measured at at least partly differing time instants. Optionally, different respiratory values may be weighted with different weighting factors, for example based on or in accordance with one or more predefined criteria. For instance, weights that are smaller or larger for time instants further away from the time of determining the troponin value may be used.

In an example, the at least one troponin value, the at least one respiratory rate value and/or the anion gap value may be time-related values. For example, the set of patient data may include time information indicative of a time of determining one or more of the at least one troponin value, the respiratory rate value and/or the anion gap value. In an exemplary implementation one or more of the at least one troponin value, the at least one respiratory rate value and/or the anion gap value may be time-stamped or may include a time stamp indicative of the time of determination of the respective parameter or subject value. As used herein, a time or time information may include an indication about one or both a day of a particular year and a time of the day.

According to an embodiment, the at least one MACE score is a numerical measure indicative of the risk for the MACE event occurring at the patient within a predetermined period of time, for example within a predetermined period of time around the time of determining the at least one troponin value, the patient-specific risk for MACE and/or the at least one MACE score. For example, the at least one MACE score may be indicative of the patient-specific risk of developing MACE within 7 days, 30 days, 60 days, or within 90 days of one or more of the assessment of a cardiac event at the hospital, receipt of at least a subset of the set patient data at the computing device, the determination of the at least one troponin value, and the determination of the at least one MACE score. Optionally, a plurality of MACE scores may be determined, wherein each MACE score may be assigned to or associated with the patient-specific risk of developing MACE within a corresponding predetermined period of time around one or more of the assessment of a cardiac event at the hospital, receipt of at least a subset of the set of patient data at the computing device, the determination of the at least one troponin value and the determination of the plurality of MACE scores.

In an exemplary implementation, the at least one MACE score may be a numerical measure indicative of the risk for a MACE event occurring at the patient within 30 days from a time of receipt of at least a subset of the set of patient data at the computing device or from a time of determining the at least one troponin value.

According to an embodiment, the obtained set of patient data further includes at least one of a gender value indicative of a sex of the patient, a demographic value indicative of a race of the patient, a white blood cell count value of the patient, and a Calcium concentration value of the patient. Therein, the at least one MACE score may be determined based on at least one of the gender value, the demographic value, the white blood cell count value, and the Calcium concentration value of the patient. One or more of the gender value, the demographic value, the white blood cell count value of the patient, and the Calcium concentration value of the patient may be used in addition to the at least one troponin value, the age and at least one of the respiratory rate value and the anion gap value to determine or compute the at least one MACE score and/or to determine, prognose or assess the patient-specific risk for MACE. Taking one or more of the aforementioned parameter values into consideration can further improve accuracy of the at least one MACE score, thereby further improving risk stratification.

Generally, the gender value, the demographic value, the white blood cell count value of the patient, and the Calcium concentration value of the patient may, be received and/or processed by the computing device sequentially in arbitrary order or sequence, or at least a subset of these parameter values may be received and/or processed by the computing device simultaneously or concurrently to compute the at least one MACE score.

According to an embodiment, the obtained set of patient data further includes the gender value, the demographic value, the white blood cell count value, and the Calcium concentration value of the patient, wherein the at least one MACE score is determined based on the gender value, the demographic value, the white blood cell count value, and the Calcium concentration value of the patient.

According to an embodiment, the method may further comprise obtaining and/or receiving, at the computing device, at least a subset of the gender value, the demographic value, the white blood cell count value of the patient, and the Calcium concentration value of the patient. For example, at least a subset of the gender value, the demographic value, the white blood cell count value of the patient, and the Calcium concentration value of the patient may be sequentially obtained and/or received at the computing device. Alternatively or additionally, the computing device may be configured to compute at least one MACE score, for example an updated MACE score, upon receipt of any one of the aforementioned parameter or subject values. Accordingly, the computing device may be configured to compute an updated MACE score and/or update the value of the at least one MACE score upon receipt of one or more of the gender value, the demographic value, the white blood cell count value of the patient, and the Calcium concentration value of the patient. Alternatively or additionally, an updated MACE score may be computed upon receipt of any new, updated, changed or more current parameter value of any parameter described herein as being potentially considered in the determination of the at least one MACE score. This can include the at least one troponin value, the respiratory rate vale, the anion gap value, and any one or more parameters described in the context of the present disclosure. By computing one or more updated MACE scores in accordance with the data or parameter values available for the patient, the accuracy of the determined risk for MACE may be gradually increased, thereby gradually improving basis for decision-making for a potential further treatment or discharge of the patient. It is noted that determining an updated MACE score can include re-assessing or re-determining the patient-specific risk for MACE using a part of or all of the parameters and parameter values considered in the computation of a previous MACE score or risk for MACE.

According to an embodiment, the computing device may be configured to obtain and/or receive one or more updated troponin values, an updated respiratory rate and/or anion gap value, and may be configured to compute an updated MACE score. Alternatively or additionally, the method may comprise obtaining and/or receiving one or more updated troponin values, an updated respiratory rate and/or anion gap value, and determining and/or computing an updated MACE score.

According to an embodiment, the computing device may be configured to obtain and/or determine contextual information associated with the determined risk for MACE and/or the at least one MACE score. Alternatively or additionally, the method may comprise the step of obtaining and/or determining contextual information associated with the determined risk for MACE and/or the MACE score. For example, the contextual information may include information or data indicative or reflective of a computational basis applied by the computing device to determine the risk for MACE and/or the at least one MACE score. Alternatively or additionally, the contextual information may include data or information for interpreting the at least one MACE score. Exemplary contextual information may include one or more notifications, messages or other informative means, which may optionally be provided at a user interface of the computing device. For example, the computing device may generate the contextual information based on processing at least a subset of the patient data. Alternatively or additionally, the computing device may be configured to retrieve the contextual information from a database.

In an example, the contextual information may include an indication or information about the parameters used for computing the at least one MACE score. In other words, the contextual information may be indicative of one or more contributing parameters and/or about one or more contributions of one or more parameters to the determined patient-specific risk for MACE. Exemplary contributing parameters may include one or more parameter values of the set of patient data considered for the determination of the risk for MACE and/or the MACE score, such as the at least one troponin value, the respiratory rate value and/or the anion gap value. Alternatively or additionally, other parameters may be considered, for example one or more health, demographic or vital parameters of the patient. Alternatively or additionally, the contextual information may include one or more of information about the time of determination of one or more of the parameters used, about a time evolution of the determined risk and/or MACE score, clinical data, scientific data or any other data potentially supporting or assisting in a decision-making process based on or using the at least one MACE score.

In yet another example, the computing device may provide the MACE score and the contextual information may include information related to the scale of the MACE score and an overall assessment of the determined risk for MACE. For instance, the computing device may output at a user interface that “the risk of a Major Adverse Cardiac Event (AMI, death and the need for cardiac revascularization procedures) within the next 30 days is assessed as low with a risk score of 1 on a scale of 1 to 10”. Optionally, the parameters that contributed to increasing the MACE score and/or the parameters that contributed to decreasing the MACE score may be provided as contextual information at the user interface.

According to an embodiment, obtaining and/or receiving the set of patient data with the at least one troponin value includes, obtaining and/or receiving a first troponin value determined at the patient at a first time, and obtaining and/or receiving a second troponin value determined at the patient at a second time different than the first time. Accordingly, a plurality of troponin values may be received at the computing device, which troponin values may be associated with different times or time instances, for example different measurement times. Optionally, the different troponin values may be based on different types of troponin tests. It should be noted that also more than two troponin value may be received at the computing device. Also, it should be noted that the plurality of troponin values may be received sequentially or simultaneously at the computing device. Taking a plurality of troponin values into consideration for the determination of the at least one MACE score can further improve risk stratification.

Optionally, the computing device may be configured to determine the at least one MACE score based on comparing the received first and second troponin values. For example, the computing device may be configured compute or calculate a difference, e.g. a delta, between and/or a temporal rate of change of the first and second troponin values, which may be indicative of a time evolution of the troponin concentration.

According to an embodiment, determining the at least one MACE score includes determining a first MACE score based on the received first troponin value, and determining a second MACE score based on both the received first troponin value and the received second troponin value. Therein, the second MACE score may refer to an updated MACE score for which both the first and second troponin values have been considered for the determination of the patient-specific risk of developing MACE. Alternatively or additionally, a difference between, a delta between and/or a rate of change (or temporal change rate) of the first and second troponin values may be considered for computing the second or updated MACE score.

According to an embodiment, the first MACE score is determined based on or using a first machine learning model of the computing device configured to compute the MACE score based on obtaining and/or receiving a single troponin value as input data. Accordingly, the first machine learning model or algorithm may be configured to determine the at least one MACE score, for example the first MACE score, based on the received first troponin value, the age, at least one of the respiratory rate value and the anion gap vale, and optionally one or more further parameters as described hereinabove and hereinbelow.

Alternatively or additionally, the second MACE score may be determined based on or using a second machine learning model of the computing device configured to compute the MACE score based on obtaining and/or receiving one or two troponin values as input data from the set of patient data, and optionally one or more further parameter values from the set of patient data. The first machine learning model and the second machine learning model may be trained using the same or different sets of training patient data but using different input data of the set of patient data, this is the first troponin value in case of the first machine learning model and one or both the first and second troponin values in case of the second machine learning model. Optionally, different parameters from the set of patient data may be considered for the computation of the MACE score in the first and second machine learning models.

Generally, the second machine learning model may allow for considerably low or erroneous troponin values, for example first troponin values. This may, for example, allow to capture or accurately determine the risk for MACE for patients having no or a low first troponin value, but having a high second troponin value. Applying the first machine learning model to such patient, and hence basing the computation of the MACE score on a single troponin value, may in certain instances result in an under-estimation of the risk for MACE. Hence, using two different machine learning model for single troponin values and for a plurality of troponin values may significantly increase the safety profile and improve risk stratification.

It should be noted that also more than two different machine learning models may be used. For example, a particular or dedicated machine learning model may be applied or invoked by the computing device to incorporate or consider historic patient data, for example indicative of a prior history of medical disease and/or treatment associated with the patient. Non-limiting examples of historic patient data may include data indicative of one or more of prior history of renal disease, prior history of cardiac disease, one or more surgeries performed on the patient or any other clinical or diagnostic data.

In an exemplary implementation, the second MACE score is determined upon obtaining and/or receiving the second troponin value. For instance, receipt of the second troponin value may trigger the computing device to invoke the second machine learning model to compute the second or updated MACE score. Optionally, a corresponding output, for example at a user interface of the computing device, may be updated, optionally along with corresponding contextual information.

In an example, the method may further comprise updating the determined first MACE score based on the determined second MACE score, thereby providing a final or updated MACE score for the patient. For instance, the second MACE score may constitute the final or updated MACE score. One or more of the determined first, second and final or updated MACE scores may optionally be stored at a data storage, and for example included in an electronic health record associated with patient.

In yet another exemplary implementation, the method may further comprise computing one or more of a difference between and a rate of change of the troponin concentration over time based on the first troponin value and the second troponin value, wherein the at least one MACE score may be determined based on one or more of the difference between, and the computed rate of change of troponin concentration over time. By taking the difference and/or the rate of change of the troponin concentration over time into account, also a development of the MACE score or risk for MACE may be determined over time.

The difference between and/or the rate of change of troponin concentration over time may be computed based on the first and second troponin values and optionally one or more further troponin values, wherein the troponin values may be measured at different time points. Time intervals between the first, the second and optionally one or more further troponin values may be the same or different. For instance, troponin values may be determined by measuring blood samples according to a predefined protocol, such as every one or two hours, or troponin values may be determined at arbitrary time intervals. Regardless of the time period between consecutively determined troponin values, the determined troponin values may optionally be normalized with respect to the time interval between the current and the previous measurement of the troponin value to compute the at least one MACE score.

In an embodiment, the method comprises obtaining and/or receiving a plurality of consecutively or sequentially determined or measured troponin values for the patient, and computing a temporal change rate of troponin in the patient and/or determining a change of troponin in the patient over time based on the received plurality of troponin values.

In an exemplary embodiment, the method may comprise risk stratifying the patient, for example ruling in or ruling out the patient as being at risk of MACE. Alternatively or additionally, the method may comprise ruling in or ruling out the patient as having a high risk of MACE. Therein, determining a risk of MACE in the patient may include determining a risk of MACE or MACE score above or exceeding a risk threshold or cut-off value for the risk or MACE score, e.g., an upper risk threshold or cut-off value. In other words, a patient may be ruled in as being at risk for MACE based on determining a high risk or probability for MACE, for example a risk of MACE or MACE score above about 60%, above about 70%, above about 80%, or above about 90%. Determining such risk for MACE or MACE score may also be indicated when a patient has been diagnosed with an acute myocardial infarction or other health-relevant cardiac event, which the patient may potentially suffer from at the time of assessment of the risk of MACE.

Alternatively or additionally, a patient may be ruled out because they are determined to have a low risk of MACE based on determining a risk of MACE or MACE score below a threshold or cut-off value for the risk or score, e.g., below a lower threshold or cut-off value. In other words, a patient may be ruled out as being at risk for MACE based on determining a low risk or probability for MACE or MACE score, for example a risk of MACE or MACE score below about 20%, 10%, 5%. 4%, 3%, 2%, or 1%.

According to an embodiment, the set of patient data further includes one or more of a Hematocrit value, an erythrocyte mean corpuscular hemoglobin concentration value, historic patient data indicative of a cardiac disease or treatment of the patient, a Creatinine value of the patient, data related to differential blood measurements, an Eosinophil concentration value, a Neutrophil concentration value, data related to immature red blood cells, a complete blood count for the patient, metabolic data of the patient, data related to a complex metabolic panel, vital data of the patient, a heart rate value for the patient, a blood pressure value for the patient, a body temperature value for the patient, historic patient data indicative of a renal disease or treatment of the patient, an Albumin concentration value, and Blood Urea Nitrogen test data of the patient. Any one or more of the aforementioned parameter values may be considered simultaneously or sequentially to compute one or more MACE scores, as described hereinabove and hereinbelow. Generally, any one or more of the aforementioned parameter values may be considered in arbitrary sequence or order, for example dependent on the sequence of receipt or availability of the corresponding patient data or parameter values.

According to an aspect of the present disclosure, there is provided a computing device or system including one or more processors for data processing, wherein the computing device is configured to carry out steps of the method, as described hereinabove and hereinbelow.

The computing device may optionally comprise a data storage, for example storing at least a part of the set of patient data data derived therefrom, the at least one MACE score and/or information or data related thereto. Alternatively or additionally, software instructions or one or more computer programs may be stored at the data storage, which when executed by one or more processors of the computing device, may instruct the computing device to perform steps of the method described hereinabove and hereinbelow.

According to an embodiment, the computing device includes one or more machine learning models, for example implemented in a classifier circuitry or logic of the computing device, as described in more detail hereinabove and hereinbelow.

The computing device described herein may refer to any data processing device, including a standalone computing device or server and computing networks with a plurality of inter-operating computing devices, such as a cloud computing system or server system. Alternatively or additionally, the computing device may be embodied, at least in part, as mobile device, such as a smart phone a tablet computer, a notebook or the like.

The computing device may, in an example, comprise one or more communication interfaces configured to communicate with one or more remote devices, for example an external data storage or database, and/or one or more remote computing devices. For instance, at least a part of the set of patient data may be received via the communication interface from an external data storage. Alternatively or additionally, the computed at least one MACE score or information related thereto may be transmitted to one or more remote devices or stored at the external data storage.

A further aspect of the present disclosure relates to a computer program, which, when executed by one or more processors of a computing device, instructs the computing device to perform steps of the method, as described hereinabove and hereinbelow.

Yet another aspect of the present disclosure relates to a computer-readable medium, for example a non-transitory computer-readable medium, storing a computer, which, when executed by one or more processors of a computing device, instructs the computing device to perform steps of the method, as described hereinabove and hereinbelow.

These and other aspects of the disclosure will be apparent from and elucidated with reference to the appended figures, which may represent exemplary embodiments.

### BRIEF DESCRIPTION OF THE DRAWINGS

The subject-matter of the present disclosure will be explained in more detail in the following with reference to exemplary embodiments which are illustrated in the attached drawings, wherein:

FIG. 1 shows a computing device or system for determining, for a patient, a risk for MACE according to an exemplary embodiment; and

FIG. 2 shows a flow chart illustrating a method of determining, for a patient, a risk for MACE according to an exemplary embodiment.

The figures are schematic only and not true to scale. In principle, identical or like parts are provided with identical or like reference symbols in the figures.

### DETAILED DESCRIPTION OF EXEMPLARY EMBODIMENTS

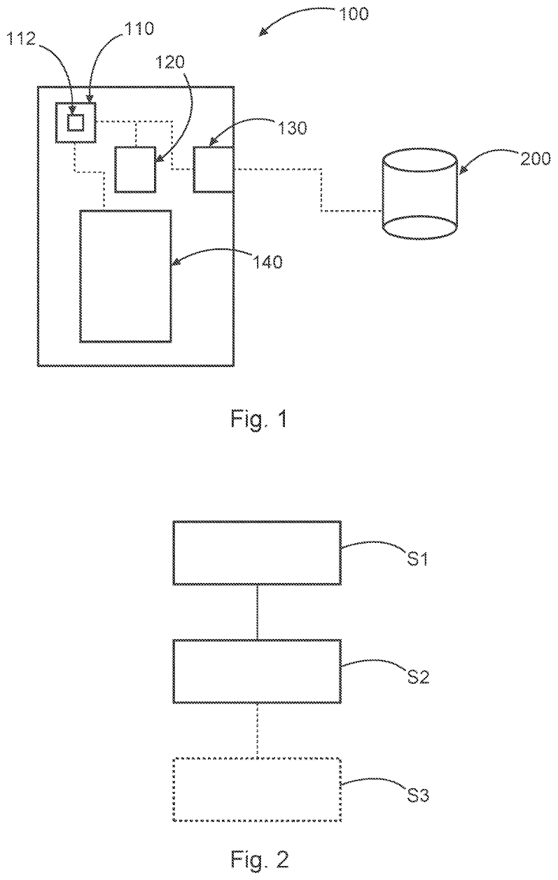

FIG. 1 shows a computing device or system 100, for example a clinical decision support system 100, configured to determine, for a patient, subject and/or individual, the risk for MACE according to an exemplary embodiment. Alternatively or additionally, the computing device 100 may be configured to determine a patient-specific risk for MACE and/or a patient-specific MACE score. Alternatively or additionally, the computing device 100 may be configured to estimate and/or prognose the risk for MACE.

The computing device 100 comprises a processing circuitry 110 or control circuitry 110 with one or more processors 112 for data processing. Optionally, the processing circuitry 110 or control circuitry 110 may include a classifier or a classifier circuitry. The computing device 100 further comprises at least one data storage 120 for storing data and/or software instructions.

The exemplary computing device 100 of FIG. 1 further comprises at least one communication circuitry or interface 130 for communicatively coupling the computing device 100 to one or more external data sources 200 that may optionally store data and/or provide data to the computing device 100. The communication circuitry 130 may be configured for wired or wireless communication with the at least one external data source 200. It should be noted that the computing device 100 may comprise a plurality of communication circuits 130 or interfaces 130 for communicatively coupling the computing device 100 to a plurality of different external data sources 200.

The one or more external data sources 200 may for example be associated with one or more external servers communicatively coupled to the computing device 100, for example via the Internet, a LAN connection, a wireless connection or a wired connection. For example, the computing device 100 may be communicatively couplable to a hospital information system, a laboratory information system, a server of a health care provider, or any other server or data processing device.

As discussed in detail hereinabove and hereinbelow, the computing device 100 may be configured to determine the risk of MACE in a patient and/or may be configured to determine the patient-specific risk for developing MACE. Therein, the computing device 100 receives a set of patient data indicative of a) at least one troponin value of the patient, b) an age of the patient, and c) at least one of a respiratory rate value of the patient and an anion gap value of the patient. Further, the computing device 100 is configured to determine, based on processing the obtained set of patient data, at least one MACE score indicative of the risk for a MACE event occurring at the patient.

Optionally, the computing device 100 may include one or more machine learning algorithms or models configured to process at least a part of the obtained set of patient data and compute the at least one MACE score, optionally along with an uncertainty for the risk and/or contextual information related to and/or explanative to the determined at least one MACE score.

In particular, a plurality of machine learning models may be implemented at the computing device 100, which may be invoked in dependence of the parameters or subject values of the set of patient data available.

In an example, a first troponin value may be received at the computing device 100 that may be used to compute a first MACE score using a first machine learning model trained to compute the first MACE score based on obtaining and/or receiving a single troponin value as input data along with the age of the patient and at least one of the respiratory rate value and the anion gap value.

Alternatively or additionally, the computing device 100 may receive a second troponin value determined at the patient at a second time different than a first time, at which the first troponin value was determined. The computing device 100 may then invoke a second machine learning model or algorithm configured or trained to compute a MACE score based on processing one or two troponin values as input data, for example along with age of the patient and at least one of the respiratory rate value and the anion gap value. For instance, the computing device 100 may be configured to compute a second MACE score upon obtaining and/or receiving the second troponin value, based on or using the first troponin value, the second troponin value, optionally the delta between the first and second troponin value, the age of the patient and at least one of the respiratory rate value and the anion gap value.

The second machine learning model may be trained to use a first and second troponin value, and optionally a delta between the first and second troponin values and/or a rate of change of the troponin value. The first machine learning model may be trained to use a single troponin value only.

Optionally, the computing device 100 may compute a change of troponin concentration over time based on comparing the first and second troponin values. The change of troponin over time may for example be computed based on dividing the difference of the first and second troponin values by the time difference between the first and second time, which may correspond to the measurement times of the first and second troponin values.

Further optionally, the computing device 100 may be configured to compute the anion gap value, for example based on obtaining and/or receiving a Sodium ion concentration value and subtracting one or both a Chloride ion concentration value and a Bicarbonate ion concentration value therefrom.

In an exemplary implementation, the obtained set of patient data may be indicative of both the respiratory rate value and the anion gap value of the patient, and both the respiratory rate value and the anion gap value may be used to compute the at least one MACE score, for example the first and/or second MACE score.

Optionally, the respiratory rate value may be indicative of a mean respiratory rate of the patient during a period of time around or about a time of determining the at least one troponin value of the patient, and the computing device 100 may be configured to compute the mean respiratory rate value.

Optionally, other or additional parameters or parameter values (or variables) may be used by the computing device 100 to determine the risk of MACE in the patient and/or the at least one MACE score. For instance, the computing device 100 may be configured to receive and/or process one or more parameter values determined based on a blood analysis. e.g. measured in the course of a complete blood count and/or in a differential blood analysis. Non-limiting blood parameters that may be used to compute the MACE score include one or more of a Hematocrit value, an erythrocyte mean corpuscular hemoglobin concentration value, mean corpuscular volume, a white blood cell concentration value, red blood cell concentration value, an Eosinophil concentration value, a Neutrophil concentration value, data related to immature red blood cells, a red blood cell distribution width value or others.

Alternatively or additionally, one or more parameters or parameter values that are determined based on metabolic tests may be considered by the computing device 100 to compute one or more MACE scores. For instance, one or more of a Blood Urea Nitrogen value, a Creatinine concentration value, a Sodium concentration value, a Potassium concentration value, a Calcium concentration value, and the anion gap value may be considered.

Alternatively or additionally, one or more parameters or parameter values that are related to vital signs of the patient may be considered to compute one or more MACE scores. For example, one or more of a mean respiratory rate value, a mean diastolic blood pressure value, a heart rate value, and a body temperature value may be considered.

Optionally, other or additional variables and/or indicators may be used by the computing device 100 to determine the risk of MACE in the patient. For instance, one or more demographic values or variables, such as age, gender, and race may be used by the computing device 100 for determining the risk of MACE. Alternatively or additionally, clinical variables, such as a type of a disorder or comorbidity, a finding, a symptom, a procedure performed on the patient, a laboratory finding, a medication, and a disease indicator, such as a diabetes indicator, a hypertension indicator or an indicator for abnormal diastolic, may be used by the computing device 100 for determining the risk of MACE and/or the at least one MACE score. Alternative or additional parameters may indicate abnormal lipids, abnormal cholesterol (LDL or HDL cholesterol), a catheterization of the patient or any other procedure or treatment performed on the patient.

In the following, non-limiting examples of subject values, patient data and/or parameters that may be taken into consideration by the computing device 100 for determining the risk of MACE are summarized: Gender, blood pressure, age, age group, erythrocyte mean corpuscular hemoglobin, atrial fibrillation. pH value, glomerular filtration rate, Oxyhemoglobin per Hemoglobin, neutrophils per 100 leukocytes, electrolyte value, magnesium value, potassium value, erythrocytes nucleated per 100 leukocytes, abnormal systolic, natriuretic peptide, urea nitrogen, electrocardiogram, anion gap, eosinophils per 100 leukocytes, chronic obstructive lung disease, chemical metabolic function tests, and partial thromboplastin time.

Other non-limiting examples of subject values, patient data and/or parameters that may be taken into consideration by the computing device 100 for determining the risk of MACE are summarized in the following: Congestive heart failure, abnormal electrolyte, abnormal magnesium, abnormal potassium, electrocardiogram, race, atrial fibrillation, gender, urinary tract infectious disease, age, age group, erythrocyte mean corpuscular hemoglobin, glomerular filtration rate, anemia, end stage renal disease, acute renal failure syndrome, abnormal phosphate, hypertension, pH value, abnormal systolic, pneumonia, hypothyroidism, hypoglycemic events, chemical metabolic function tests, coronary artery bypass grafting, and neutrophils per 100 leukocytes.

Optionally, the computing device 100 may be configured to determine one or more of the patient having a prior history of cardiac disease, and the patient having a prior history of renal disease, for example based on processing historic patient data indicative of a medical disease history of the patient.

For instance, the computing device 100 may be configured to determine that the patient has a prior history of renal disease based on determining an estimated Glomerular Filtration Rate value, eGFR, and/or a creatinine value, e.g., an eGFR value below a predefined eGFR threshold value and/or a creatinine value below a predefined creatinine threshold value, recorded for the patient in the historic patient data. Exemplary threshold values for eGFR may be about 40 ml/min/1.73 m² to about 80 ml/min/1.73 m², for example about 50 ml/min/1.73 m² to about 70 ml/min/1.73 m². In particular, the eGFR threshold value may be about 60 ml/min/1.73 m². Exemplary threshold values for Creatinine may be about 1.1 mg/dL to about 1.4 mg/dL, for example about 1.3 mg/dL.

Alternatively or additionally, the computing device 100 may be configured to determine that patient has a prior history of cardiac disease based on processing historic patient data indicative of a medical disease history of the patient and determining a troponin value within a predetermined range and/or above a predetermined threshold value for troponin reported for the patient. For example, a troponin concentration value above about 50 ng/L and/or a rate of change of troponin of greater than about 15 ng/L/h reported for the patient may indicate that the patient has a prior history of cardiac disease.

The computing device 100 further includes a user interface 140 for receiving one or more user inputs. For instance, one or more subject values or patient data may be provided to the computing device 100 via the user interface 140.

The user interface 140 may be configured to provide or output information to a user. For example, the computing device 100 may be configured to display the at least one MACE score and/or generated information indicative of the likelihood or risk of MACE and/or contextual information at the user interface 140.

FIG. 2 shows a flow chart illustrating a method of determining a patient-specific risk for developing MACE according to an exemplary embodiment, for example using a computing device 100 as described with reference to FIG. 1.

Step S1 comprises obtaining and/or receiving, at the computing device 100, a set of patient data indicative of a) at least one troponin value of the patient, b) an age of the patient, and c) at least one of a respiratory rate value of the patient and an anion gap value of the patient. One or more of these subject values or patient data may be received by the computing device 100 based on retrieving the one or more subject values from the data storage 120 and/or from one or more external data sources 200.

Step S2 comprises determining, based on processing the obtained set of patient data with the computing device 100, at least one MACE score indicative of the risk for a MACE event occurring at the patient.

It is noted that one or more further optional steps, as described hereinabove in the summary part and with reference to FIG. 1, may be performed for determining the risk of MACE in the patient and/or for determining one or more MACE scores. For example, in an optional step S3 the at least one MACE score and/or information indicative of a likelihood for the patient having or suffering from MACE may optionally be displayed at the user interface 140 of the computing device 100.

In an exemplary implementation, the computing device 100 may include one or more machine learning models. The one or more machine learning models may particularly be trained and/or configured to determine the one or more MACE scores, for example based on processing the set of patient data and/or receiving the set of patient data as input data.

The one or more machine learning models can be trained using a set of training patient data, which may for example be obtained from a set of electronic health records (EHR) or EHR data of a plurality of patients. For instance, EHR data can be collected from a plurality of health care providers across one or more nations. The EHR data can be aggregated and further processed to generate the training patient data.

For example, the training patient data may be obtained from the EHR data based on extracting data being associated with a particular patient and being indicative of one or more of the parameters considered as input for the one or more machine learning models, such as the at least one troponin value, the age of the patient, the respiratory rate value, the anion gap value of the patient and/or any other parameter described herein to be potentially usable for computing the at least one MACE score, such as the gender value.

The EHR data may further include an indication about one or more diseases a patient suffered from in the past. For instance, diagnosis codes, such as according to the International Statistical Classification of Diseases and Related Health Problems, so-called ICD codes, can be stored in the EHR data. Based on the diagnosis codes, it can be determined whether MACE occurred in a particular patient in the past, such that a corresponding label for MACE can be generated and included in the training patient data.

The training patient data obtained from the EHR data may optionally be cleaned for subsequent processing, for example based on removing erroneous data records. Further optionally, the training patient data may be split into a training dataset, which can be used for optimizing the one or more machine learning models, and a test dataset, which can be used for validation of the one or more machine learning models. For instance, model parameters of the one or more machine learning models can be tuned or adjusted using the training dataset to determine optimized model parameters, which may then be used to validate the one or more machine learning models using the test dataset. The process can be repeated iteratively, for example until the one or more machine learning models satisfy a target performance on the test dataset.

In the following, an exemplary implementation of a machine learning model and aspects of training such model are summarized. The machine learning model may be based on or be a gradient boosting algorithm, but any other artificial intelligence-based (AI-based) algorithm, such as an artificial neural network, a feed forward neural network and/or a deep neural network, can be used instead.

The training patient data may, for example, be represented as

$${\{ ( {x}_{i} , {y}_{i} ) \}}_{i = 1}^{n} ,$$

with i=1 . . . n being the number of patients or EHR records considered in the training patient data, x, being a vector indicative of the parameters considered as input for the machine learning model, and yᵢ being indicative of the label for MACE, and optionally for a MACE score. Exemplary numbers of patients or individual patient records n considered in the training patient data may range from several thousands to several hundreds of thousands, such as for example about one hundred thousand. A differentiable loss function L (y, F (x)), such as for example cross entropy loss or other loss function, may be used to iteratively determine model parameters of the machine learning model F (x) in a number of M iterations.

For training, the initial model F₀ may be initialized with a constant value for the model parameters, and mathematically expressed as

$${F}_{0} ( x ) = {arg min}_{γ} {\sum }_{i = 1}^{n} L ( {y}_{i} , γ ) .$$

For each iteration m=1 . . . . M, the so-called pseudo residuals may be computed as

$${r}_{im} = - {[ \frac{\partial L ( {y}_{i} , F ( {x}_{i} ) )}{\partial F ( {x}_{i} )} ]}_{F ( x ) - {F}_{m - 1} ( x )} , for i = 1 , … , n .$$

Further, in each iteration, a base learner hₘ (x) may be fit to the computed pseudo-residuals, and a multiplier γₘ may be computed by solving the following one-dimensional optimization problem:

$${γ}_{m} = {arg min}_{γ} {\sum }_{i = 1}^{n} L ( {y}_{i} , {F}_{m - 1} ( {x}_{i} ) + γ {h}_{m} ( x ) ) .$$

As an example, decision tree may be used as base learner, for example with a total number of about 200 trees and a maximum depth of about four for each tree.

Further, in each iteration, the model can be updated in accordance with the following equation:

$${F}_{m} ( x ) = {F}_{m - 1} ( x ) + γ {h}_{m} ( x ) ,$$

and the final model F_{M}(x) may then be output after M iterations, for example when the model performance meets one or more threshold criteria.

As mentioned above, gradient boosting or gradient tree boosting may optionally be utilized. For instance, the one or more machine learning models may include, be based on or implemented as gradient boosting algorithm according to the XGBoost library. Accordingly, the one or more models may be based on a scalable end-to-end tree boosting system, such as e.g. XGBoost.

XGBoost can be considered an optimized distributed gradient boosting library designed to be highly efficient, flexible and portable, thereby allowing to implement machine learning models under the Gradient Boosting framework that provide a parallel tree boosting.

While the invention has been illustrated and described in detail in the drawings and foregoing description, such illustration and description are to be considered illustrative or exemplary and not restrictive: the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

The following exemplary Aspects are provided, the numbering of which is not to be construed as designating levels of importance:
Aspect 1 provides a computer-implemented method of determining, for a patient, a risk of developing a major adverse cardiovascular event, MACE, the method comprising:
   obtaining, at a computing device, a set of patient data indicative of a) at least one troponin value of the patient, b) an age of the patient, and c) at least one of a respiratory rate value of the patient and an anion gap value of the patient; and
   determining, based on processing the obtained set of patient data with the computing device, at least one MACE score indicative of the risk for a MACE event occurring at the patient.
Aspect 2 provides the method according to Aspect 1, wherein determining the at least one MACE score includes computing the risk for the MACE event occurring at the patient based on at least one machine learning model of the computing device configured to receive and process at least a subset of the patient data as input data.
Aspect 3 provides the method according to any one of Aspects 1-2, wherein the anion gap value is indicative of a difference between Sodium ion concentration and one or both Chloride ion concentration and Bicarbonate ion concentration.
Aspect 4 provides the method according to any one of Aspects 1-3, further comprising:
   obtaining a Sodium ion concentration value and at least one of a Chloride ion concentration value and a Bicarbonate ion concentration value of the patient; and
   computing the anion gap value based on the received Sodium ion concentration value and at least one of the Chloride ion concentration value and the Bicarbonate ion concentration value of the patient.
Aspect 5 provides the method according to any one of Aspects 1-4, wherein the obtained set of patient data is indicative of both the respiratory rate value and the anion gap value of the patient.
Aspect 6 provides the method according to any one of Aspects 1-5, wherein the respiratory rate value is indicative of a mean respiratory rate of the patient during a period of time about a time of determining the at least one troponin value of the patient.
Aspect 7 provides the method according to any one of Aspects 1-6, wherein the at least one MACE score is a numerical measure indicative of the risk for the MACE event occurring at the patient within a predetermined period of time.
Aspect 8 provides the method according to any one of Aspects 1-7, wherein the at least one MACE score is a numerical measure indicative of the risk for a MACE event occurring at the patient within 30 days from a time of receipt of at least a subset of the set of patient data or from a time of determining the at least one troponin value.
Aspect 9 provides the method according to any one of Aspects 1-8, wherein the obtained set of patient data further includes at least one of a gender value indicative of a sex of the patient, a demographic value indicative of a race of the patient, a white blood cell count value of the patient, and a Calcium concentration value of the patient; and
   wherein the at least one MACE score is determined based on at least one of the gender value, the demographic value, the white blood cell count value, and the Calcium concentration value of the patient.
Aspect 10 provides the method according to Aspect 8, wherein the obtained set of patient data further includes the gender value, the demographic value, the white blood cell count value, and the Calcium concentration value of the patient; and/or
   wherein the MACE score is determined based on the gender value, the demographic value, the white blood cell count value, and the Calcium concentration value of the patient.
Aspect 11 provides the method according to any one of Aspects 1-10, wherein obtaining the set of patient data including the at least one troponin value includes:
   obtaining a first troponin value determined at the patient at a first time; and
   obtaining a second troponin value determined at the patient at a second time different than the first time.
Aspect 12 provides the method according to Aspect 11, wherein determining the at least one MACE score includes:
   determining a first MACE score based on the received first troponin value; and
   determining a second MACE score based on both the received first troponin value and the received second troponin value.
Aspect 13 provides the method according to Aspect 12, wherein the first MACE score is determined based on a first machine learning model of the computing device configured to compute the MACE score based on obtaining a single troponin value as input data.
Aspect 14 provides the method according to any one of Aspects 12-13, wherein the second MACE score is determined based on a second machine learning model of the computing device configured to compute the MACE score based on obtaining one or two troponin values as input data.
Aspect 15 provides the method according to any one of Aspects 12-14, wherein the second MACE score is determined upon obtaining the second troponin value.
Aspect 16 provides the method according to any one of Aspects 12-15, further comprising:
   updating the determined first MACE score based on the determined second MACE score, thereby providing an updated MACE score for the patient.
Aspect 17 provides the method according to any one of Aspects 11-16, further including:
   computing a rate of change of troponin concentration based on the first troponin value and the second troponin value; and
   wherein the at least one MACE score is determined based on the computed rate of change of troponin concentration.
Aspect 18 provides the method according to any one of Aspects 1-17, wherein the obtained set of patient data further includes one or more of a Hematocrit value, an erythrocyte mean corpuscular hemoglobin concentration value, historic patient data indicative of a cardiac disease or treatment of the patient, a Creatinine value of the patient, data related to differential blood measurements, an Eosinophil concentration value, a Neutrophil concentration value, data related to immature red blood cells, a complete blood count for the patient, metabolic data of the patient, data related to a complex metabolic panel, vital data of the patient, a heart rate value for the patient, a blood pressure value for the patient, a body temperature value for the patient, historic patient data indicative of a renal disease or treatment of the patient, an Albumin concentration value, and Blood Urea Nitrogen test data of the patient.
Aspect 19 provides a computing device including one or more processors for data processing, wherein the computing device is configured to carry out steps of the method according to any one of Aspects 1-18.
Aspect 20 provides a computer program, which, when executed by one or more processors of a computing device, instructs the computing device to perform steps of the method according to any one of Aspects 1-18.
Aspect 21 provides a non-transitory computer-readable medium storing a computer program according to Aspect 20.
Aspect 22 provides the method, computing device, computer program, or non-transitory computer-readable medium of any one or any combination of Aspects 1-21 optionally configured such that all elements or options recited are available to use or select from.

1. A computer-implemented method of determining, for a patient, a risk of developing a major adverse cardiovascular event, MACE, the method comprising:
obtaining, at a computing device, a set of patient data indicative of a) at least one troponin value of the patient, b) an age of the patient, and c) at least one of a respirator\hat{ }′ rate value of the patient and an anion gap value of the patient; and determining, based on processing the obtained set of patient data with the computing device, at least one MACE score indicative of the risk for a MACE event occurring at the patient.

2. The method according to claim 1, wherein determining the at least one MACE score includes computing the risk for the MACE event occurring at the patient based on at least one machine learning model of the computing device configured to receive and process at least a subset of the patient data as input data.

3. The method according to claim 1, wherein the anion gap value is indicative of a difference between Sodium ion concentration and one or both Chloride ion concentration and Bicarbonate ion concentration.

4. The method according to claim 1, further comprising:
obtaining a Sodium ion concentration value and at least one of a Chloride ion concentration value and a Bicarbonate ion concentration value of the patient; and computing the anion gap value based on the received Sodium ion concentration value and at least one of the Chloride ion concentration value and the Bicarbonate ion concentration value of the patient.

5. The method according to claim 1, wherein the obtained set of patient data is indicative of both the respiratory rate value and the anion gap value of the patient.

6. The method according to claim 1, wherein the respiratory rate value is indicative of a mean respiratory rate of the patient during a period of time about a time of determining the at least one troponin value of the patient.

7. The method according to claim 1, wherein the at least one MACE score is a numerical measure indicative of the risk for the MACE event occurring at the patient within a predetermined period of time.

8. The method according to claim 1, wherein the at least one MACE score is a numerical measure indicative of the risk for a MACE event occurring at the patient within 30 days from a time of receipt of at least a subset of the set of patient data or from a time of determining the at least one troponin value.

9. The method according to claim 1, wherein the obtained set of patient data further includes at least one of a gender value indicative of a sex of the patient, a demographic value indicative of a race of the patient, a white blood cell count value of the patient, and a Calcium concentration value of the patient; and
wherein the at least one MACE score is determined based on at least one of the gender value, the demographic value, the white blood cell count value, and the Calcium concentration value of the patient.

10. The method according to claim 8, wherein the obtained set of patient data further includes the gender value, the demographic value, the white blood cell count value, and the Calcium concentration value of the patient; and/or
wherein the MACE score is determined based on the gender value, the demographic value, the white blood cell count value, and the Calcium concentration value of the patient.

11. The method according to claim 8, wherein obtaining the set of patient data including the at least one troponin value includes:
obtaining a first troponin value determined at the patient at a first time; and obtaining a second troponin value determined at the patient at a second time different than the first time.

12. The method according to claim 11, wherein determining the at least one MACE score includes:
determining a first MACE score based on the received first troponin value; and
determining a second MACE score based on both the received first troponin value and the received second troponin value.

13. The method according to claim 12, wherein the first MACE score is determined based on a first machine learning model of the computing device configured to compute the MACE score based on obtaining a single troponin value as input data.

14. The method according to claim 12, wherein the second MACE score is determined based on a second machine learning model of the computing device configured to compute the MACE score based on obtaining one or two troponin values as input data.

15. The method according to claim 12, wherein the second MACE score is determined upon obtaining the second troponin value.

16. The method according to claim 12, further comprising: updating the determined first MACE score based on the determined second MACE score, thereby providing an updated MACE score for the patient.

17. The method according to claim 11, further including: computing a rate of change of troponin concentration based on the first troponin value and the second troponin value; and
wherein the at least one MACE score is determined based on the computed rate of change of troponin concentration.

18. The method according to claim 1, wherein the obtained set of patient data further includes one or more of a Hematocrit value, an erythrocyte mean corpuscular hemoglobin concentration value, historic patient data indicative of a cardiac disease or treatment of the patient, a Creatinine value of the patient, data related to differential blood measurements, an Eosinophil concentration value, a Neutrophil concentration value, data related to immature red blood cells, a complete blood count for the patient, metabolic data of the patient, data related to a complex metabolic panel, vital data of the patient, a heart rate value for the patient, a blood pressure value for the patient, a body temperature value for the patient, historic patient data indicative of a renal disease or treatment of the patient, an Albumin concentration value, and Blood Urea Nitrogen test data of the patient.

19. A computing device including one or more processors for data processing, wherein the computing device is configured to carry out steps of the method according to claim 1.

20. A computer program, which, when executed by one or more processors of a computing device, instructs the computing device to perform steps of the method according to claim 1.

21. A non-transitory computer-readable medium storing a computer program according to claim 20.
